(19) United States
(12) Patent Application Publication
(10) Pub. No.: US 2005/0192543 A1
(43) Pub. Date: Sep. 1, 2005

(54) Reciprocating syringe

(76) Inventor: Wilmer L. Sibbitt, Albuquerque, NM (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 11/056,558

(22) Filed: Feb. 11, 2005

Related U.S. Application Data
(60) Provisional application No. 60/544,688, filed on Feb. 13, 2004.

(51) Int. Cl. A61M 5/315

(52) U.S. Cl. 604/218

(57) ABSTRACT

A reciprocating syringe is described. The syringe includes a syringe barrel with a syringe plunger with a thumb rest for injecting or aspirating fluid and reciprocating member with thumb a rest which move in a guide track and a reciprocating device connecting the syringe plunger to the reciprocating member so that when the syringe plunger moves in one direction the reciprocating member moves in the opposite direction.

### RELATED APPLICATIONS

This application claims priority to provisional application Ser. No. 60/544,688 filed Feb. 13, 2004.

### BRIEF DESCRIPTION

The present invention relates generally to syringes which can be operated with one hand, and more particularly to reciprocating syringes.

### BACKGROUND OF THE INVENTION

Syringes are an essential element in the day-to-day practice of medicine and nursing, but are also essential in industry, laboratory science, research, and animal husbandry. Syringes are used to inject medications, to aspirate body fluids, to provide vacuum, and to transfer fluids. The syringe design most commonly used in medicine consists of a barrel made of plastic and an internal plunger which is moved into or out of the barrel, resulting in pressure or a vacuum, respectively. The difference in pressure between the volume in the syringe and the outside environment are produced by movement of the plunger, resulting in movement of fluid into or out of the syringe. These differences in pressure create the desired effect of a syringe, that is, aspiration or injection.

One-handed aspiration with a standard syringe is difficult and awkward, resulting in loss of fine control and power during aspiration. With loss of control, there is a higher rate of procedure failure and contamination. With loss of power, speed of aspiration is impaired, especially for viscous fluids. Because of the loss of strength and control with one-handed aspiration, procedures that demand either fine control of the syringe during aspiration or the generation of a powerful vacuum require the use of both hands during aspiration to maintain both strength and control.

The ability to use a syringe with one hand so that the other hand can be used for other tasks is important in many complicated medical procedures. First attempts at one-handed aspiration involved the use of an external apparatus which is integral with the syringe and allows the plunger to be advanced or retracted using squeezing motions using one hand rather than pulling motion using two hands. For details of this approach, refer to U.S. Pat. No. 3,990,446. It has been proposed for an aspirating syringe to have a plunger guide for a reciprocating plunger assembly. For more details of this approach, refer to U.S. Pat. No. 5,582,595. Another proposed approach is to use an adapter which can be fitted on a conventional syringe to provide an assembly for aspirating tissue. For more details of this approach, refer to U.S. Pat. No. 5,135,511. It has also been proposed to provide a syringe with an external slide, which attaches to the plunger, that permits one-handed operation. Refer to U.S. Pat. No. 4,484,915 or U.S. Pat. No. 4,639,248 for details.

Other proposed structures describe more complicated two compartment syringes and double plunger systems, but these are usually based on a single barrel and are intended to mix or administer two different substances. Refer to U.S. Pat. No. 3,685,514 or U.S. Pat. No. 5,188,616 for details. Double piston devices, either mechanically or hydraulically driven, have been proposed for aspirating fluids or administering medications. Refer to U.S. Pat. No. 4,036,232 or U.S. Pat. No. 4,437,859 for details.

Reciprocating syringes are syringes with two plungers or plunger-equivalents where the plungers are mechanically linked by a line, filament, cord, gears, pulley systems or hydraulics. Such syringes, both single and double barrel, may be fitted with combinations of one-way valves, so that the syringes have utility as pumps, lavage devices, or irrigation devices, U.S. Pat. No. 6,245,046.

### OBJECTS AND SUMMARY OF THE INVENTION

It is an object of the present invention to provide a reciprocating syringe of the type described in U.S. Pat. No. 6,245,046, which brings the accessory plunger closer to the axis of the functional syringe barrel.

A first embodiment of the present invention provides a reciprocating syringe with a syringe barrel which includes an opening at one end through which fluid may be ejected or aspirated, a syringe plunger within the syringe barrel for forcing or aspirating fluid through said syringe opening, a reciprocating member which moves in a guide track disposed in said syringe barrel and a reciprocating device connecting the syringe plunger to the reciprocating member so that when the syringe plunger moves in one direction the reciprocating device moves in the opposite direction.

Another embodiment of the present invention provides a reciprocating syringe with the syringe barrel which includes an opening at one end through which fluid may be forced or aspirated; a first syringe plunger sliding within the syringe barrel for forcing fluid through the syringe barrel opening; a second auxiliary barrel surrounding said first barrel providing a space therebetween; a reciprocating member which moves in a guide track disposed in said space; and a reciprocating device connecting the first syringe plunger to the reciprocating device so that when the syringe plunger moves in one direction, the reciprocating device moves in the opposite direction and vice versa.

In another aspect of the present invention, the guide track is disposed above the syringe barrel.

Other objects and features of the present invention will be apparent from the following detailed description of the preferred embodiments.

### BRIEF DESCRIPTION OF THE DRAWINGS

The invention will be described in conjunction with the accompanying drawings in which:

FIG. 1 is a schematic view of the first embodiment of the syringe device with guide track for the reciprocating member internal to the syringe barrel;

FIG. 2 is a sectional view taken along the line 2-2 of FIG. 1;

FIG. 3 is a top view of the syringe stopper;

FIG. 4 is an end view of the syringe device showing the thumb rests;

FIG. 5 is a sectional view of a syringe showing another embodiment of the invention;

FIG. 6 is a view of the syringe stopper for the syringe of FIG. 5;

FIG. 7 is an end view of the syringe of FIG. 5 showing the thumb rests;

FIGS. 8 and 9 are schematic views of a syringe showing another embodiment of the invention;

FIG. 10 is an end view of the embodiment of FIG. 8; and

FIG. 11 is a sectional view taken along the line 11-11 of FIG. 8.

### DETAILED DESCRIPTION OF THE PREFERRED EMBODIMENTS

For the purpose of this invention the term “axial direction of a syringe” refers to the line along or parallel to the center axis of the syringe.

For the purpose of this invention the term “proximal” refers to the direction toward the user of a syringe, and for the purpose of this invention the term “distal” refers to the direction away from the user of the syringe.

For the purpose of this invention the term “reciprocating member” refers to plungers or sliders, which are connected to a syringe plunger by a reciprocating device and moves in a direction opposite to that of the syringe plunger to which the reciprocating member is connected.

For the purpose of this present invention a “reciprocating device” refers to a device which combines the function of connecting a syringe plunger to a reciprocating member and causes the direction of motion of the syringe plunger and the reciprocating member to be in opposite direction. Examples of such devices include a connecting cord running over a smooth edge of the syringe wall, a connecting cord which runs through a pulley mounted on a pulley post.

For the purpose of this invention the term “track” refers to any means which constrains the motion of a reciprocating member such as a hollow tube in which the reciprocating member slides, or a post on which the reciprocating member slides.

The present invention provides a syringe that permits injection and aspiration of fluids or gases using one hand, with applications in health care research, animal husbandry and the industry. The present invention utilizes fundamental changes in syringe design. In one embodiment the syringe of the present invention includes an internal reciprocating member or accessory plunger which is mechanically associated with the plunger of the functional syringe, resulting in a set of reciprocating plungers. Thus when one plunger is depressed with the thumb, the syringe injects, and when the accessory plunger is pressed with the thumb, the syringe aspirates. This arrangement permits the index and middle fingers to stay in one position during aspiration and injection while the thumb only need move laterally to the alternate plunger in order to change the direction of flow (i.e., aspiration or injection). The resulting syringe is highly stable, since only the thumb position changes and then because of the close proximity of the accessory plunger and the syringe plunger it need only move a short lateral distance. The syringe of the present invention can be used in all cases where standard syringes are used. The syringe has particular use in medical procedures when single-handed injection/aspiration is required such as cardiac catheterization, emergency procedures, certain type of surgeries, pediatrics and veterinary procedures and those handicapped individuals who can only use one hand. The use of this new design is expected to be large.

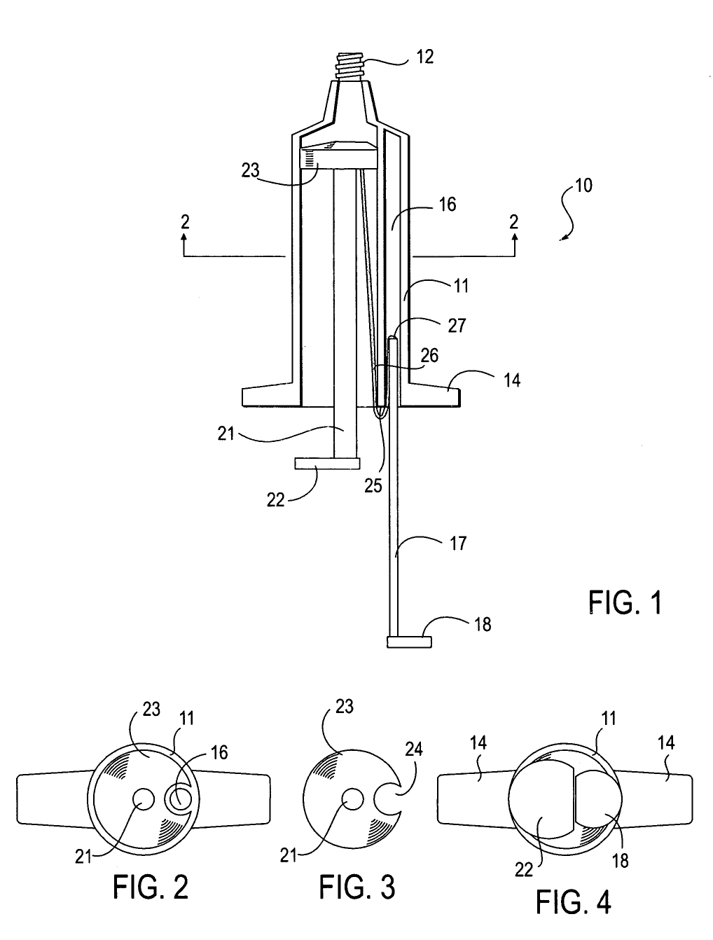

Referring particularly to FIGS. 1-4, the syringe 10 of one preferred embodiment is illustrated. The syringe 10 includes a syringe barrel 11 made of plastic, glass or other suitable material. A needle or cannula (not shown) is held on the end of syringe barrel 11 by a conventional needle or cannula fitting 12. Finger flanges 14 are connected to and extend outwardly from proximal end of the syringe barrel. A tube or track 16 extends along and is connected to the interior wall of the barrel. The tube provides a track for movement of a reciprocating accessory plunger 17. The accessory plunger has a thumb rest 18 at its proximal end. The syringe includes a syringe plunger 21 with a thumb rest 22 at its proximal end and a stopper 23 made from rubber or other flexible or tight sealing material at the distal end of the plunger 21. The stopper is configured with a cut out 24 so as to provide a competent seal with the internal track. The syringe barrel and guide tube or track may be integrated together by bonding, gluing, casting or other methods of integration. The shape of the auxiliary tube and shape of the accessory plunger can be round as shown or can be of any other suitable matching configuration and the stopper can be configured to seal therewith. The plunger 17 and the stopper 23 are mechanically attached by a connecting member 26 such as a cord, line, string, wire, etc., which reaches from the stopper 23 over the edge 25 of the barrel 11 and is connected to the end 27 of the plunger 17. The plunger is configured or slotted so as to provide adequate space for the connecting member to move within the tube or track. The edge of the barrel functions as a pulley device which in the present embodiment is merely a rounded edge of the wall of the tube. The above-described mechanical arrangement results in a reciprocating thumb operated double plunger device that may be operated with one hand and effect both aspiration and injection and which is well balanced because of the proximity of the plungers.

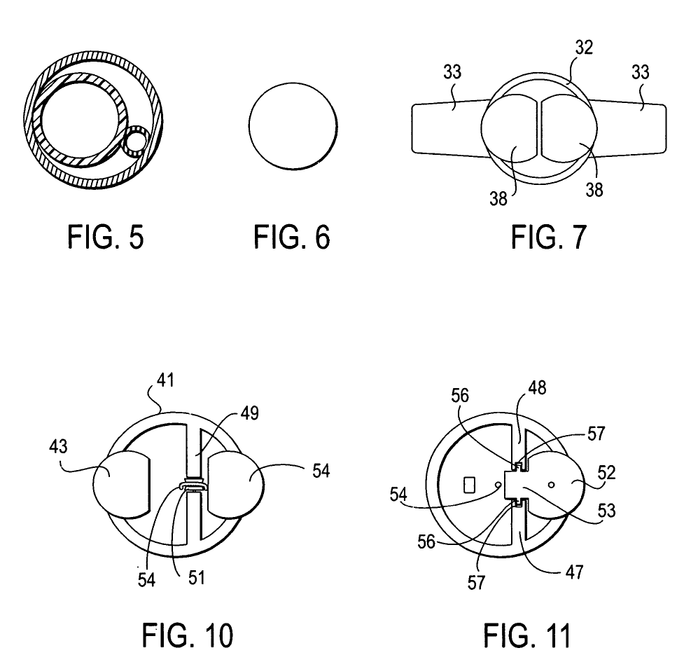

In a second embodiment in which only a sectional view and end views are illustrated in FIGS. 5-7, the main barrel or the syringe barrel 31 is surrounded by an external auxiliary barrel 32 to which the finger flanges 33 are attached and which provides a space 34 for the auxiliary plunger tube or track 36. In this embodiment the stopper 37 is of conventional design. Thumb rests 38 and 39 are in close proximity requiring only slight lateral movement of the thumb to go between injection to aspiration.

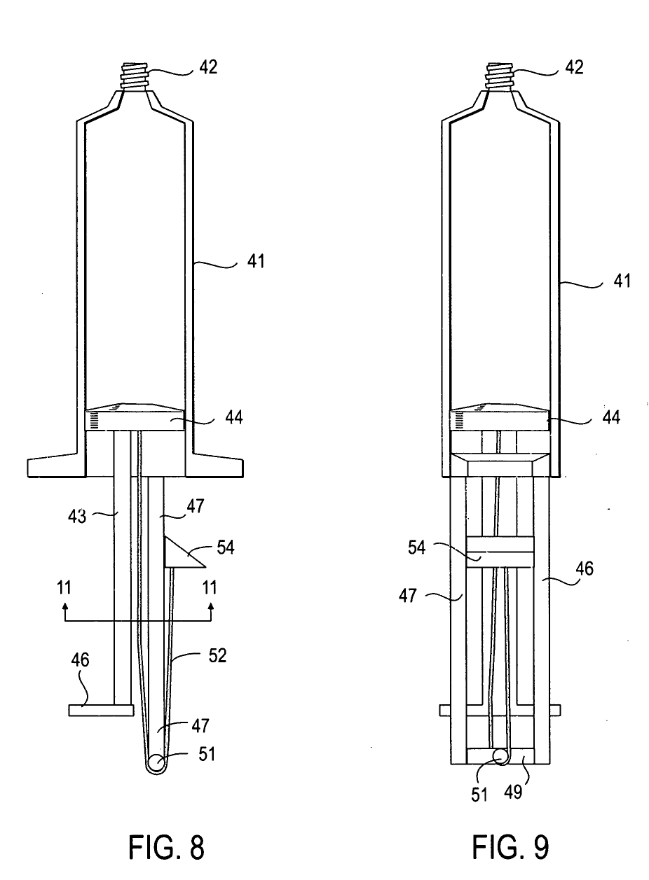

FIGS. 8-11 illustrate another embodiment of the invention. The syringe includes syringe barrel 41 with the needle fitting 42 at one end, a plunger 43 with stopper 44 and thumb rest 46. In this embodiment a pseudo-plunger is provided. The pseudo-plunger may comprise a pair of spaced posts 47, 48 attached to and extending proximity from the syringe barrel 41 and terminating in a cross member 49 which may include a pulley 51. The pseudo plunger includes a thumb rest 52 and is attached to a fitting 53 which guides the plunger along the spaced posts or tracks. A drive line 54 is connected between the stopper, reeled over the pulley 51 and attached to the thumb rest 52 of the pseudo plunger. The thumb rest fitting 53 is adopted to engage the posts as for example by having ears 56 which ride in guide slots 57 formed in the posts. The post serve as a track for guiding the pseudo plunger in its reciprocating movement.

The various embodiments described provide a mechanical arrangement with reciprocating thumb operated plungers. The single barrel arrangement provides a more stable syringe in which the finger rests are closer to each other for a more comfortable grasp. The thumb rests are also closer together requiring a smaller lateral movement of the thumb during reciprocating operation of the syringe. This provides for stability of the syringe during operation.

1. A balanced reciprocating syringe comprising:
a barrel with finger flanges extending outwardly at the proximal end of the barrel;
a syringe plunger having a thumb rest at its proximal end and a stopper at its other end; and
a reciprocating member having a thumb rest and positioned so that it moves on an axis parallel to the syringe axis;
a guide track having its axis parallel to and within the syringe barrel for receiving and guiding the reciprocating member; and
means mechanically linking syringe plunger and reciprocating member to one another whereby they reciprocate in opposite directions in response to pressure on one or the other of the thumb rests.

2. A balanced reciprocating syringe as in claim 1 in which the barrel is the syringe barrel the guide track is disposed within the syringe barrel, the stopper is shaped to accommodate the track, and the reciprocating member comprises an auxiliary plunger.

3. A balanced reciprocating syringe as in claim 1 including an syringe barrel disposed within the barrel, the guide track is between the syringe barrel and the barrel, and the reciprocating member comprises a plunger.

4. A balanced reciprocating syringe as in claim 1 in which the reciprocating member comprises:
a pair of spaced posts extend proximately from the barrel to provide a track; and
the reciprocating member comprises a thumb rest and fitting for guiding the thumb rest along the spaced posts.
